(19) United States
(12) Patent Application Publication
(10) Pub. No.: US 2011/0087047 A1
(43) Pub. Date: Apr. 14, 2011

(54) Vinyl acetate production process

(76) Inventors: Noel Hallinan, Loveland, OH (US); Wayne J. Brtko, Glen Mills, PA (US); Brian A. Salisbury, Oxford, PA (US)

(21) Appl. No.: 12/587,580

(22) Filed: Oct. 9, 2009

(51) Int. Cl. C07C 67/00

(52) U.S. Cl. 560/261

(57) ABSTRACT

A process for the production of vinyl acetate is disclosed. The process comprises reacting ethylene, acetic acid, and oxygen in the presence of a catalyst to produce a reaction mixture comprising vinyl acetate, ethylene, carbon dioxide, acetic acid and ethylene glycol diacetate. The reaction mixture is separated to a gas stream comprising ethylene, oxygen, and carbon dioxide and a crude vinyl acetate stream comprising vinyl acetate, acetic acid and ethylene glycol diacetate. An ethylene glycol diacetate stream, which comprises at least 50 wt % of ethylene glycol diacetate, is isolated from the crude vinyl acetate stream and hydrolyzed to recover acetic acid. Recovered acetic acid may be recycled to the acetoxidation reaction.

### FIELD OF THE INVENTION

The invention relates to the preparation of vinyl acetate. More particularly, the invention relates to recovering acetic acid from ethylene glycol diacetate.

### BACKGROUND OF THE INVENTION

Vinyl acetate is commonly produced by the reaction of ethylene, oxygen and acetic acid in the presence of a palladium-gold catalyst. See, for example, U.S. Pat. No. 3,743,607. Palladium and gold are expensive precious metals. Therefore, many efforts have been made to increase the catalytic activity and reduce the amount of catalyst needed. For example, U.S. Pat. No. 6,022,823 teaches calcining the support impregnated with palladium and gold salts prior to reducing the metals. The catalyst shows improved activity.

The acetoxidation of ethylene to vinyl acetate is commonly performed in a gas phase, fixed bed tubular reactor. Vinyl acetate is recovered by condensation and scrubbing, and purified by distillation. Unreacted ethylene, oxygen and acetic acid are recovered by distillation and recycled to the acetoxidation.

In addition to vinyl acetate, the acetoxidation produces a number of byproducts, including carbon dioxide and ethylene glycol diacetate. Carbon dioxide is primarily produced by the combustion of ethylene and vinyl acetate. Carbon dioxide is removed from the reaction product mixture by distillation and absorption with a potassium carbonate solution.

The acetoxidation produces from 1 to 2 pounds of ethylene glycol diacetate, which is currently disposed of, per 100 pounds of vinyl acetate. Ideally, acetic acid is recovered from ethylene glycol diacetate and reused in the acetoxidation.

### SUMMARY OF THE INVENTION

The invention is a process for the production of vinyl acetate. The process comprises reacting ethylene, acetic acid and oxygen in the presence of a catalyst to produce a reaction mixture comprising vinyl acetate, ethylene, carbon dioxide, acetic acid and ethylene glycol diacetate. The reaction mixture is separated to a gas stream comprising ethylene and carbon dioxide and a crude vinyl acetate stream comprising vinyl acetate, acetic acid and ethylene glycol diacetate. An ethylene glycol diacetate stream, which preferably comprises at least 50 wt % of ethylene glycol diacetate, is isolated from the crude vinyl acetate stream and hydrolyzed to recover acetic acid. Recovered acetic acid is preferably recycled to the acetoxidation.

### DETAILED DESCRIPTION OF THE INVENTION

The process of the invention comprises reacting ethylene, acetic acid, and oxygen in the presence of a catalyst. The acetoxidation is preferably performed in a gas phase, fixed bed tubular reactor using a supported catalyst. Preferably, the acetoxidation is performed at a temperature within the range of 150° C. to 250° C., more preferably 175° C. to 200° C. Preferably, the acetoxidation is performed under a pressure within the range of 50 psia to 150 psia, and more preferably within the range of 70 psia to 140 psia.

The amount of oxygen in the combined feed to the reactor is determined by many factors. Preferably, the amount of oxygen in the combined feed is within the range of 5 mol % to 15 mol %, more preferably within the range of 5 mol % to 12 mol %. Acetic acid may be introduced into the reactor in liquid form or in vapor form. Preferably, the amount of acetic acid in the combined feed is within the range of 10 mol % to 20 mol %. Preferably, the amount of ethylene in the combined feed is within the range of 65 mol % to 80 mol %. Preferably, ethylene, oxygen and acetic acid are mixed and the mixture is then fed into the reactor as a gas.

Suitable catalysts include those known to the vinyl acetate industry. Preferably, the catalyst is a palladium-gold catalyst. Methods for preparing palladium-gold catalysts are known. For instance, U.S. Pat. No. 6,022,823, the teachings of which are incorporated herein by reference, teaches how to prepare a palladium-gold catalyst which has high activity and selectivity. Preferably, the palladium-gold catalyst is supported on an inorganic oxide. Preferably, the inorganic oxide is selected from the group consisting of alumina, silica, titania, the like, and mixtures thereof.

Preferably, the supported catalysts have palladium contents from 0.1 wt % to 3 wt % and gold contents from 0.1 wt % to 3 wt %. More preferably, the catalysts contain from 0.5 wt % to 1.5 wt % of palladium and from 0.25 wt % to 0.75 wt % of gold. The weight ratio of palladium to gold is preferably within the range of 5:1 to 1:3 and more preferably within the range of 2.5:1 to 1:1.5.

The reaction mixture is withdrawn from the reactor and separated into a gas stream and a crude vinyl acetate stream. The gas stream comprises ethylene, oxygen and carbon dioxide. The crude vinyl acetate stream comprises vinyl acetate, acetic acid and ethylene glycol diacetate. Carbon dioxide is separated by distillation or absorption from ethylene which is then recycled to the acetoxidation reactor. The crude vinyl acetate stream is separated to an ethylene glycol diacetate stream and a vinyl acetate product stream. The vinyl acetate product stream may be subjected to further purification to produce vinyl acetate with a desired purity.

The ethylene glycol diacetate stream comprises ethylene glycol diacetate and acetic acid. It may also comprise other components such as ethylene glycol and polyvinyl acetate. The ethylene glycol diacetate stream preferably comprises at least 50 wt % of ethylene glycol diacetate. More preferably, the ethylene glycol diacetate stream comprises from 50 wt % to 95 wt % of ethylene glycol diacetate and from 5 wt % to 50 wt % of acetic acid. Most preferably, the ethylene glycol diacetate stream comprises from 70 wt % to 80 wt % of ethylene glycol diacetate and from 20 wt % to 30 wt % of acetic acid.

The ethylene glycol diacetate stream undergoes hydrolysis to convert ethylene glycol diacetate to acetic acid and ethylene glycol. The hydrolysis is preferably performed in the presence of a base catalyst. Suitable base catalysts include ammonia, organic amines, metal hydroxides, the like and mixture thereof. Preferred base catalysts are potassium hydroxide and sodium hydroxide. The hydrolysis product comprises acetic acid and ethylene glycol which are preferably then separated by distillation. Resultant acetic acid is preferably recycled to the acetoxidation and ethylene glycol is isolated as a byproduct.

Preferably, the hydrolysis is performed in a waste acid stripper of an acetic acid production process. Alternatively, the hydrolysis is performed in a hydrolysis reactor and the hydrolyzed products are combined with a heavy ends distillation column bottoms stream from an acetic acid production process and the combined streams are distilled in a waste acid stripper of the acetic acid process.

Acetic acid production and heavy ends distillation therewith are well known in the industry. For instance, U.S. Pat. No. 7,345,197, the teachings of which are incorporated herein by reference, discloses producing acetic acid by methanol carbonylation. In the process, an acetic acid product stream is usually withdrawn from the reactor and is separated by a flash separation into a liquid fraction comprising the catalyst and catalyst stabilizer, and a vapor fraction comprising the acetic acid product, the reactants and impurities generated during the carbonylation reaction. The liquid fraction is then recycled to the carbonylation reactor. The vapor fraction is passed to a light ends distillation which separates the vapor fraction to an overhead comprising methyl iodide, water, methanol, methyl acetate, and light impurities and an acetic acid stream comprising acetic acid, a small amount of water, and heavy impurities. The acetic acid stream is passed to a drying column to remove water and is consequently subjected to a heavy ends distillation to remove the heavy impurities such as propionic acid.

The heavy ends distillation bottoms stream preferably comprises from 90 wt % to 98 wt % of acetic acid and from 2 wt % to 10 wt % of propionic acid. More preferably, the heavy ends distillation bottom stream comprises from 93 wt % to 96 wt % of acetic acid and from 4 wt % to 7 wt % of propionic acid. The hydrolysis product and the heavy ends distillation bottoms stream can be combined in any ratio, depending on the design and capacity of the waste acid stripper.

The waste acid stripper is preferably a packed distillation column having 6 or more theoretical stages. The waste acid stripper is preferably operated at an overhead temperature from 150° C. to 160° C. and overhead pressure from 35 psia to 45 psia. The overhead stream of the waste acid stripper comprises preferably greater than 90 wt %, more preferably greater than 95 wt %, and most preferably greater than 98 wt %, of acetic acid. The overhead stream can be recycled to the acetoxidation reaction of the vinyl acetate process. Alternatively, the overhead stream can be recycled to the heavy ends distillation column of the acetic acid process.

The waste acid stripper is preferably operated at a bottom temperature from 190° C. to 210° C. and a bottom pressure from 40 psia to 45 psia. The bottoms stream of the waste acid stripper comprises preferably greater than 50 wt %, more preferably greater than 60 wt %, and most preferable greater than 75 wt %, of ethylene glycol. The bottoms stream can be disposed of or subjected to further separation to recover ethylene glycol as a byproduct.

The following example is merely illustrative. Those skilled in the art will recognize many variations that are within the spirit of the invention and scope of the claims.

### Example

This example is modeled by a computer program. Feed to a heavy ends distillation column (HEC) of an acetic acid production process is a mixture (2.6 parts by weight) which comprises 600 ppm of water, 99.89% of acetic acid, and 500 ppm of propionic acid. The HEC has 38 theoretical stages.

An ethylene glycol diacetate stream (0.5 parts by weight) from a vinyl acetate process is fed to a base catalyzed hydrolysis reactor along with water (0.1 parts by weight). The ethylene glycol diacetate stream comprises 0.2% of water, 29.8% of acetic acid, 69% of glycol diacetate and 1% of polyvinyl acetate. The reactor operates at 43 psia and 157° C. A product stream (0.6 parts by weight) from the reactor comprises 0.2% of water, 73.9% of acetic acid, 25.1% of ethylene glycol and 0.8% of polymer. This stream is fed to the WAS along with the above HEC bottoms stream and an additional water stream (0.1 parts by weight).

The WAS overhead stream (3 parts by weight), which comprises 2.3% of water, 86.8% of acetic acid and 10.9% of propionic acid, is taken at 41 psia and 156° C. This stream is recycled to the HEC for recovery of acetic acid. The WAS bottom stream (0.3 parts by weight) comprises 0.2% of water, 25.1% of acetic acid, 12.1% of propionic acid, 61% of ethylene glycol and 1.6% of polymer. This stream is taken at 43 psia and 203° C.

We claim:

1. A process for the production of vinyl acetate comprising: (a) reacting ethylene, acetic acid, and oxygen in the presence of a catalyst to produce a reaction mixture comprising vinyl acetate, ethylene, carbon dioxide, acetic acid and ethylene glycol diacetate; (b) separating the reaction mixture to form a gas stream comprising ethylene and carbon dioxide and a crude vinyl acetate stream comprising vinyl acetate, acetic acid and ethylene glycol diacetate; (c) isolating an ethylene glycol diacetate stream from the crude vinyl acetate stream; and (d) hydrolyzing the ethylene glycol diacetate stream and recovering acetic acid.

2. The process of claim 1, further comprising recycling acetic acid from step (d) to the reaction of step (a).

3. The process of claim 1, wherein the ethylene glycol diacetate stream comprises at least 50 wt % of ethylene glycol diacetate.

4. The process of claim 1, wherein the ethylene glycol diacetate stream comprises from 5 wt % to 50 wt % of acetic acid and from 50 wt % to 95 wt % of ethylene glycol diacetate.

5. The process of claim 1, wherein the ethylene glycol diacetate stream comprises from 20 wt % to 30 wt % of acetic acid and from 70 wt % to 80 wt % of ethylene glycol diacetate.

6. The process of claim 1, wherein the hydrolysis of the ethylene glycol diacetate stream is performed in the presence of a base catalyst.

7. The process of claim 1, wherein step (a) is performed in a gas phase, fixed bed tubular reactor using a supported catalyst.

8. The process of claim 7, wherein the supported catalyst comprises a palladium, gold, an inorganic oxide support.

9. The process of claim 8, wherein the inorganic oxide support is selected from the group consisting of alumina, silica, titania and mixtures thereof.

10. The process of claim 7, wherein step (a) is performed at a temperature within the range of 150° C. to 250° C. and a pressure from 50 psia to 150 psia.

11. The process of claim 1, wherein the acetic acid of step (d) is recovered in a waste acid stripper of an acetic acid production process.

12. The process of claim 11, wherein in step (d) the ethylene glycol diacetate stream is hydrolyzed in a waste acid stripper of an acetic acid production process.
